(19) United States
(12) Patent Application Publication
(10) Pub. No.: US 2018/0117043 A1
(43) Pub. Date: May 3, 2018

(54) USE OF SRC PROTEIN INHIBITOR IN THE MANUFACTURE OF A MEDICAMENT FOR THE PROPHYLAXIS AND/OR TREATMENT OF ALZHEIMER'S DISEASE

(71) Applicant: JenKem Technology Co., Ltd. (Beijing), Beijing (CN)

(72) Inventors: Zewang FENG, Beijing (CN); Jinliang WANG, Beijing (CN); Yan LIU, Beijing (CN); Xuan ZHAO, Beijing (CN)

(21) Appl. No.: 15/848,918

(22) Filed: Dec. 20, 2017

Related U.S. Application Data
(63) Continuation of application No. PCT/CN2016/085701, filed on Jun. 14, 2016.

(30) Foreign Application Priority Data
Jun. 24, 2015 (CN) 201510355573.0

(51) Int. Cl. A61K 31/506; A61K 31/496; A61P 25/28

(52) U.S. Cl. CPC A61K 31/506; A61P 25/28; A61K 31/496

(57) ABSTRACT

The present invention provides use of a Src protein inhibitor N-(2-chloro-6-methylphenyl)-2-{6-[4-(3-hydroxyethyl)-1-piperazinyl]-2 -methyl-4-pyrimidinylamino}-5-thiazolecarboxamide (formula I) in the manufacture of a medicament for the prophylaxis and/or treatment of Alzheimer's disease. An Aβ42 oligomer and a tested drug were applied to primary isolated fetal mouse cerebral cortical neuron for 24 h to test the efficacy of the compound of formula I at different concentrations against Aβ42 oligomer toxicity in vitro, and the results show that the compound of formula I has a wide range of effective concentration and high cell viability at effective concentration.

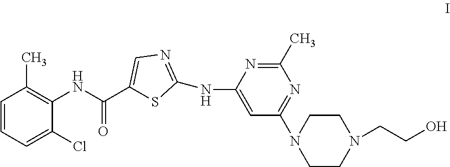 [SMILES: Cc1nc(Nc2ncc(C(=O)Nc3c(C)cccc3Cl)s2)cc(N2CCN(CCO)CC2)n1]

### CROSS-REFERENCE TO RELATED APPLICATION

This application is a continuation application of International patent application No. PCT/CN2016/085701, filed on Jun. 14, 2016, which claims the benefit and priority of Chinese patent application No. CN201510355573.0, filed on Jun. 24, 2015, each of which is incorporated herein by reference in its entirety and for all purposes.

### FIELD OF THE INVENTION

The present invention relates to use of a Src protein inhibitor in the manufacture of a medicament for the prophylaxis and/or treatment of Alzheimer's disease, in particular to compound N-(2-chloro-6-methylphenyl)-2-{6-[4-(3-hydroxyethyl)-1-piperazinyl]-2 -methyl-4-pyrimidinylamino}-5-thiazolecarboxamide in the manufacture of a medicament for the prophylaxis and/or treatment of Alzheimer's disease.

### BACKGROUND OF THE INVENTION

Senile dementia (also known as Alzheimer's disease, AD) is a common central nervous system degenerative disease, the patients first suffer from recent memory disorders clinically, followed by persistent intelligence recession, loss of judgment and reasoning ability, and movement disorder, etc. As early as 1976, Davies et al discovered the phenomenon of dysfunction and apoptosis in central neurons and synapses particularly those involved in cholinergic neurons in AD patients. With the development of molecular biology, genes associated with AD have been discovered including APP (amyloid precursor protein), PS1 (presenilin-1), PS2 (presenilin-2), ApoE4 and mitochondrial-encoded cytochrome oxidase I and II, etc. The main pathological changes of AD are a regional neuronal loss, brain tissue atrophy, appearance of a large number of amyloid plaques, also known as senile plaques (SP), and neurofibritary tangles deposition. The senile plaques are surrounded by dystrophic axones, activated spongiocytes and astrocytes. The degree of dementia in an AD patient is positively correlated with developing of amyloid plaques. The main component of the senile plaques is β-amyloid peptide-Aβ42, which has a molecular weight of about 4.2 KD, consists of 39 to 43 amino acid residues, and is derived from the amyloid precursor protein (APP). The complete APP is Type I transmembrane protein. Proteolytic cleavage of APP begins with the cleavage of its extracellular domain under the action of α-secretase or β-secretase, resulting in the production of sAPPα(soluble APP)/sAPPβ and CTFα(C-terminal fragments)/CTFβ, and γ-secretase cleaves CTFα/CTFβ to generate a series of Aβ and CTFγ with different molecular sizes. Most of the PS mutations may result in an increased production of Aβ42 by affecting γ-secretase (PS may be a component of γ-secretase).

As shown in the investigation for health-threatening epidemics, the degree of concern for AD has leapt to the front position in senile diseases, and AD becomes a disease with a high incidence threatening the life and health of the elderly only next to cardiovascular disease, cancer, and cerebral apoplexy. Although scientists have studied AD for many years, this disease, as the most common dementia, is still incurable. The AD therapeutics currently marketed can only improve the symptoms of AD patients, slow but not prevent or reverse the progression of the disease. Therefore, the development of new drugs aimed at the etiology of AD disease not new mechanisms of symptoms is still the key research directions of domestic and foreign pharmaceutical companies.

Aβ42 plays a key role in occurrence and development of AD, and the current further researches of metabolism and toxicity of Aβ42 provide a wide range of potential drug targets for the treatment of AD. In WO2012/103282, ZHONG Yi reported a preferred target for the treatment of Alzheimer's disease-the epidermal growth factor receptor (EGFR) screened by employing transgenic fruit flies and double transgenic mice. Inhibition of EGFR could improve Aβ42-induced early memory loss in transgenic fruit flies and mice, and the pharmacological activity screening data shows that the EGFR inhibitors being studied (such as EKB-569, CL-387785, HKI-272, BIBW 2992, HKI-357, ZD-6474, AEE 788, XL647, BMS-599626, IPI-504, 17-AAG, JKF-006, JKF-011, JKF-027, GJ-06, GJ-06-1, GJ-12, and GJ-12-1) are effective in the treatment of Aβ42-induced memory loss in transgenic fruit flies and double transgenic mice expressing Aβ42. The patent screened three compounds JKF-006, JKF-011 and JKF-027 with a preferable curative effect.

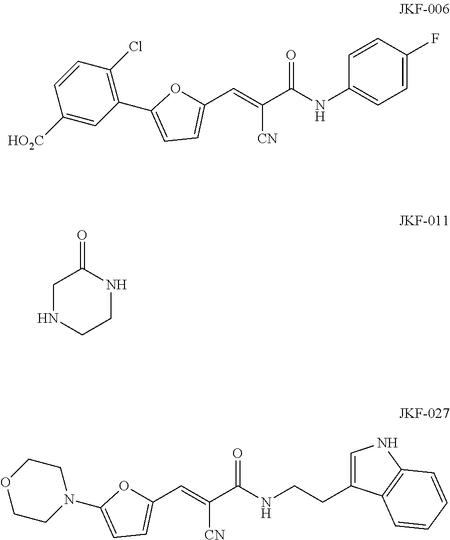 [SMILES: N#C/C(=C\c1ccc(-c2cc(C(=O)O)ccc2Cl)o1)C(=O)Nc1ccc(F)cc1 ; O=C1CNCCN1 ; N#C/C(=C\c1ccc(N2CCOCC2)o1)C(=O)NCCc1c[nH]c2ccccc12]

N-(2-chloro-6-methylphenyl)-2-{6-[4-(3-hydroxyethyl)-1-piperazinyl]-2-methy-4-pyrimidinylamino}-5-thiazolecarboxamide, namely dasatinib, is a second-generation tyrosine kinase inhibitor, which can be used for the treatment of chronic myeloid leukemia, and targets Abl, Src, c-Kit, ephrin receptors and other tyrosine kinases but does not target EGFR or Her2.

The present invention has found that Src protein inhibitors, in particular dasatinib, are useful as a medicament for the prophylaxis and/or treatment of Alzheimer's disease, and in vitro test results show that they have activity for the prophylaxis and/or treatment of Alzheimer's disease and inhibition of Aβ42 cytotoxicity significantly higher than that of the previously reported EGFR inhibitors.

### SUMMARY OF THE INVENTION

The present invention provides use of a Src protein inhibitor in the manufacture of a medicament for the treatment and/or prophylaxis of Alzheimer's disease (AD).

The present invention also provides use of a Src protein inhibitor in the manufacture of a medicament for the treatment and/or prophylaxis of nervous system degenerative diseases.

The present invention also provides use of a Src protein inhibitor in the manufacture of a medicament for cognitive disorder or learning and memory disorder and/or for improvement of cognitive disorder or learning and memory disorder.

The present invention also provides use of a Src protein inhibitor in the manufacture of a medicament for inhibition of Aβ42 cytotoxicity.

The present invention also provides use of a Src protein inhibitor in the manufacture of a medicament for treatment and/or prophylaxis of vascular dementia or vascular cognitive impairment.

The present invention also provides use of a Src protein inhibitor in the manufacture of a medicament for treatment and/or prophylaxis of cholinergic neuron degenerative diseases.

Preferably, the Src protein inhibitor according to the present invention is a Src/Abl dual-acting inhibitor. More preferably, the Src protein inhibitor according to the present invention is selected from dasatinib or a pharmaceutically acceptable salt thereof, bosutinib or a pharmaceutically acceptable salt thereof.

In a specific embodiment, the present invention provides use of N-(2-chloro-6-methylphenyl)-2-{6-[4-(3-hydroxyethyl)-1-piperazinyl]-2-methy-4-pyrimidinylamino}-5-thiazolecarboxamide (as shown by formula I) or a pharmaceutically acceptable salt thereof, a solvate thereof or a solvate of the salt, an ester or ether thereof, a precursor or metabolite thereof in the manufacture of a medicament for the treatment and/or prophylaxis of Alzheimer's disease (AD).

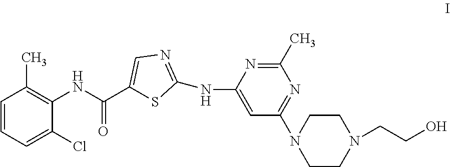 [SMILES: Cc1nc(Nc2ncc(C(=O)Nc3c(C)cccc3Cl)s2)cc(N2CCN(CCO)CC2)n1]

Furthermore, the present invention also provides use of the compound of formula I or a pharmaceutically acceptable salt thereof, a solvate thereof or a solvate of the salt, an ester or ether thereof, a precursor or metabolite thereof in the manufacture of a medicament for the treatment and/or prophylaxis of nervous system degenerative diseases.

Furthermore, the present invention also provides use of the compound of formula I or a pharmaceutically acceptable salt thereof, a solvate thereof or a solvate of the salt, an ester or ether thereof, a precursor or metabolite thereof in the manufacture of a medicament for the treatment and/or prophylaxis, improvement of cognitive disorder or learning and memory disorder.

Furthermore, the present invention also provides use of the compound of formula I or a pharmaceutically acceptable salt thereof, a solvate thereof or a solvate of the salt, an ester or ether thereof, a precursor or metabolite thereof in the manufacture of a medicament for inhibition of Aβ42 cytotoxicity.

Furthermore, the present invention also provides use of the compound of formula I or a pharmaceutically acceptable salt thereof, a solvate thereof or a solvate of the salt, an ester or ether thereof, a precursor or metabolite thereof in the manufacture of a medicament for treatment and/or prophylaxis of vascular dementia or vascular cognitive impairment.

Furthermore, the present invention also provides use of the compound of formula I or a pharmaceutically acceptable salt thereof, a solvate thereof or a solvate of the salt, an ester or ether thereof, a precursor or metabolite thereof in the manufacture of a medicament for the treatment and/or prophylaxis of cholinergic neuron degenerative diseases.

The present invention has surprisingly found that, the compound of formula I and JKF-027 reported in WO2012/103282 were used for comparative test for efficacy against Aβ42 protein mouse cerebral cortical neuron toxicity, an Aβ42 oligomer (10 μmol/L) and a tested drug with different concentrations were applied to primary isolated fetal mouse cerebral cortical neuron from Kunming mouse with pregnancy of 17 days for 24 h to test the efficacy of the compound of formula I and JKF-027 against Aβ42 oligomer toxicity in vitro, and the results show that JKF-027 only has an effective concentration range of 0.1-10 μmol/L, while the compound of formula I has an effective concentration range of 0.01-1000 μmol/L, a relatively large dose span, which is 10⁴ times that of JKF-027, and has cell viabilities which are close to each other in the effective concentration and exceed the most effective concentration of JKF-027, and has a better overall effect.

The compound of the formula I according to the present invention should be understood to comprise the amorphous substance and any crystalline forms of the compound of formula I, for example the amorphous substance and any crystalline forms of dasatinib disclosed in U.S. Pat. No. 6,596,746B, US20050215795A, WO2009053854A1, CN102086195A, and CN104341410A.

The “pharmaceutically acceptable salt” according to the present invention refers to a modified derivative obtained by forming an acid salt by a parent compound thereof. The examples of the acceptable salts include, but are not limited to, inorganic or organic acid salts. The pharmaceutically acceptable salt includes the conventional non-toxic salts formed by the parent compound, for example, salts formed by non-toxic inorganic or organic acids. For example, the conventional non-toxic salts include salts derived from inorganic acids such as hydrochloride, hydrobromide, sulfate, phosphate and nitrate, etc.; and salts derived from organic acids such as acetate, propionate, succinate, glycollate, stearate, lactate, malate, tartrate, citrate, ascorbate, pamoate, maleate, hydroxymaleate, phenylmaleate, glutamate, benzoate, salicylate, fumarate, tosylate, mesylate, oxalate and isethionate, etc. For example, a pharmaceutically acceptable salt of the compound of formula I according to the present invention refers to hydrochloride, hydrobromide, sulfate, phosphate, nitrate, acetate, propionate, succinate, glycollate, stearate, lactate, malate, tartrate, citrate, ascorbate, pamoate, maleate, hydroxymaleate, phenylmaleate, glutamate, benzoate, salicylate, fumarate, tosylate, mesylate, oxalate and isethionate of the compound of formula I.

When the compound of the present invention is basic, the salt can be prepared from a pharmaceutically acceptable non-toxic acid, including inorganic or organic acids. The acid includes acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, nitric acid, pamoic acid, pantothenic acid, phosphoric acid, succinic acid, sulfuric acid, tartaric acid and p-toluenesulfonic acid and the like.

The solvate of the present invention may be the solvate well known in the art, and the solvate of the compound of formula I may be a hydrate such as monohydrate, ethanol solvate, or isopropanol solvate, etc.

The ester or ether of the present invention may be may be an ester or an ether well known in the art, and the ester or ether of the compound of formula I may be formed as an ether or ester of the compound using conventional chemical reactions in the art.

The precursor or metabolite of the present invention may be a precursor or metabolite well known in the art, as long as the precursor or metabolite can be converted into the compound through metabolism in vivo.

In a preferred embodiment of the present invention, the compound of formula I is a compound of formula I in the form of free base, or a pharmaceutically acceptable salt thereof.

The present invention also relates to a pharmaceutical composition comprising a compound of formula I or a pharmaceutically acceptable salt thereof, a solvate thereof or a solvate of the salt, an ester or ether thereof, a precursor or metabolite thereof, and one or more pharmaceutically acceptable additives.

The pharmaceutical composition of the present invention includes any composition prepared by mixing the compound of the present invention and a pharmaceutically acceptable additive. The term “Pharmaceutically acceptable” means that the additive such as a carrier, diluent or excipient must be compatible with the other formulation ingredients and be harmless to the recipient thereof.

The compound of the present invention may be administered orally, parenterally (e.g., intramuscularly, intraperitoneally, intravenously, ICV, by intracisternal injection or infusion, subcutaneous injection or implantation), and by inhalation spray, nasal, vaginal, rectal, sublingual or topical route of administration, and they may be formulated alone or together with additives, such as conventional non-toxic and pharmaceutically acceptable carriers, adjuvants and excipients, etc., to form an unit dosage form containing a suitable dosage suitable for a variety of routes of administration.

The pharmaceutical composition of the compound of the present invention for administration may be presented in suitable dosage unit form and may be prepared by any of the well-known methods in the art of pharmacy. All methods include the step of mixing the active ingredient with one or more pharmaceutically acceptable additives. In general, the pharmaceutical composition is prepared in the following way: uniformly and intimately mixing the active ingredient with a liquid additive or a fine solid additive or both, and shaping the product into the desired formulation. The pharmaceutical composition comprises the active ingredient in an amount effective to produce a desired effect on the disease process or symptoms.

The pharmaceutical composition containing the active ingredient may be formulated into suitable oral forms such as a tablet, aqueous or oily suspension, dispersible powder or granule, emulsion, hard capsule or soft capsule, syrup, and the like. Any method well known in the art of pharmaceutical formulation may be used to prepare the composition for oral use. The composition may contain one or more additives selected from a sweetener, flavoring agent, colorant and preservative.

The tablet contains the active ingredient and a non-toxic pharmaceutically acceptable additive suitable for the manufacture of tablet. These additives may be an inert diluent (such as calcium carbonate, sodium carbonate, lactose, calcium phosphate or sodium phosphate), granulating and disintegrating agents (such as corn starch or alginic acid), binder (such as starch, gelatin or arabic gum) and lubricant (such as magnesium stearate, stearic acid or talc). The tablet may be uncoated or coated by known techniques to prolong disintegration and absorption in the gastrointestinal tract to provide a long term sustained therapeutic effect. For example, a delayed-release material (such as glyceryl monostearate or glyceryl distearate) may be used, a sustained- or controlled-release tablet may be prepared by coating, and a rapid-release tablet may be formulated, etc.

Formulation for oral use may also be hard capsules in which the active ingredient is mixed with an inert solid diluents (such as calcium carbonate, calcium phosphate or kaolin), or soft capsules, in which the active ingredient is mixed with an aqueous or oily medium (such as peanut oil, liquid paraffin or olive oil).

The aqueous suspension contains the active ingredient and a non-toxic pharmaceutically acceptable additive suitable for use in the manufacture of aqueous suspension. The additive is a suspending agent (such as sodium carboxymethylcellulose, methylcellulose, hydroxypropylmethylcellulose, sodium alginate, polyvinylpyrrolidone, arabic gum), dispersing or wetting agents (such as lecithin, polyoxyethylene stearate, heptadecylethylnenoxy hexadecanol, polyoxyethylene sorbitol monooleate). The aqueous suspension may also contain one or more preservatives (such as ethyl p-hydroxybenzoate or n-propyl p-hydroxybenzoate), one or more colorants, one or more flavoring agents and one or more sweeteners (such as sucrose or saccharin).

The oily suspension may be prepared by suspending the active ingredient in a vegetable oil (such as peanut oil, olive oil, sesame oil or coconut oil) or in a mineral oil (such as liquid paraffin). The oily suspension may contain a thickening agent (such as beeswax, hard paraffin or cetanol). The above sweetener and flavoring agent may be added, and an antioxidant (such as ascorbic acid) may be added.

The pharmaceutical composition of the present invention may also be in the form of an oil-in-water emulsifier. The oily phase may be a vegetable oil (such as olive oil or peanut oil) or a mineral oil (such as liquid paraffin) or a mixture thereof Suitable emulsifier may be a natural gum (such as arabic gum or tragacanth gum), a natural phospholipid (such as soya lecithin) and an ester or partial ester derived from fatty acid with hexitol anhydride (such as sorbitan monooleate), and a condensation product of the partial ester with ethylene oxide (such as polyoxyethylene sorbitan monooleate). The emulsifier may also contain a sweetener or flavoring agent.

The syrup can be formulated with a sweetener (such as glycerol, propylene glycol, sorbitol or sucrose). The syrup may also contain a wetting agent, preservative and flavoring agent, as well as colorant.

The pharmaceutical composition may be in the form of a sterile injectable aqueous or oily suspension. The suspension may be formulated according to a process well-known in the art using suitable dispersing or wetting agents and suspending agents as described above.

The pharmaceutical composition may also be in the form of a suppository for rectal administration. The suppository can be prepared by mixing the drug with a suitable non-irritating additive which is solid at ordinary temperatures but liquid at the rectal temperature. The additive may be cocoa butter and polyethylene glycol.

For topical application, a cream, ointment, jelly, liquid preparation or suspension, etc. containing the compound of the present invention may be employed. Similarly, a transdermal patch may also be used for topical administration.

Accordingly, the present invention further provides use of N-(2-chloro-6-methylphenyl)-2-{6-[4-(3-hydroxyethyl)-1-piperazinyl]-2-methy-4-pyrimidinylamino}-5-thiazolecarboxamide (as shown by formula I) or a pharmaceutically acceptable salt thereof, a solvate thereof or a solvate of the salt, an ester or ether thereof, a precursor or metabolite thereof in the manufacture of a medicament for the treatment and/or prophylaxis of Alzheimer's disease (AD), the medicament comprises the compound of formula I or a pharmaceutically acceptable salt thereof, a solvate thereof or a solvate of the salt, an ester or ether thereof, a precursor or metabolite thereof and one or more pharmaceutically acceptable additives.

The medicament of the present invention may be an oral preparation, intramuscular, intraperitoneal, intravenous, ICV, intracisternal injection or infusion preparations, subcutaneous injection or implantation preparations, inhalation spray, nasal, vaginal, rectal, sublingual or topical preparations.

Preferably, in the medicament, the compound of formula I or pharmaceutically acceptable salt thereof, solvate thereof or solvate of the salt, ester or ether thereof, precursor or metabolite thereof is contained in an amount of 0.1-99.9%, more preferably 1-90%, more preferably 5-80%, and most preferably 10-70% by mass percent.

### BRIEF DESCRIPTION OF THE DRAWINGS

FIG. 1 is a bar graph of the first in vitro pharmacodynamic test of Compound I against Aβ42 protein.

FIG. 2 is a bar graph of the second in vitro pharmacodynamic test of Compound I against Aβ42 protein.

FIG. 3 is a bar graph of the third in vitro pharmacodynamic test of Compound I against Aβ42 protein.

FIG. 4 is a mean bar graph of three independent repeated in vitro pharmacodynamic tests of Compound I against Aβ42 protein.

### DETAILED DESCRIPTION OF THE INVENTION

The following non-limiting examples may enable those of ordinary skill in the art to more fully understand the present invention, it must be illustrated that the following examples are intended to illustrate the present invention rather than to limit the present invention. The improvements made to the present invention according to the essence of the present invention all fall within the protection scope of the present invention. The terms described in the present invention are all scientific terms commonly used in the field of science, and do not limit the scope of the present invention in any way.

The 2-chloro-6-methylaniline used in the examples was purchased from Hangzhou Dakang Chemical Co., Ltd., (E)-3-ethoxyacryloyl chloride was purchased from Hunan Huateng Pharmaceutical Co., Ltd., 4,6-dichloro-2-methylpyrimidine from Changzhou Ruisheng Chemical Co., Ltd., 1-(2-hydroxyethyl) piperazine was purchased from Shanghai HATCH Chemical Co., Ltd., and other reagents were purchased from Sinopharm Chemical Reagent Beijing Co., Ltd.

### EXAMPLE 1

### Preparation of N-(2-chloro-6-methylphenyl)-2-{6-[4-(3-hydroxyethyl)-1-piperazinyl]-2-methyl-4-pyrimidinylamino}-5-thiazolecarboxamide (Compound I)

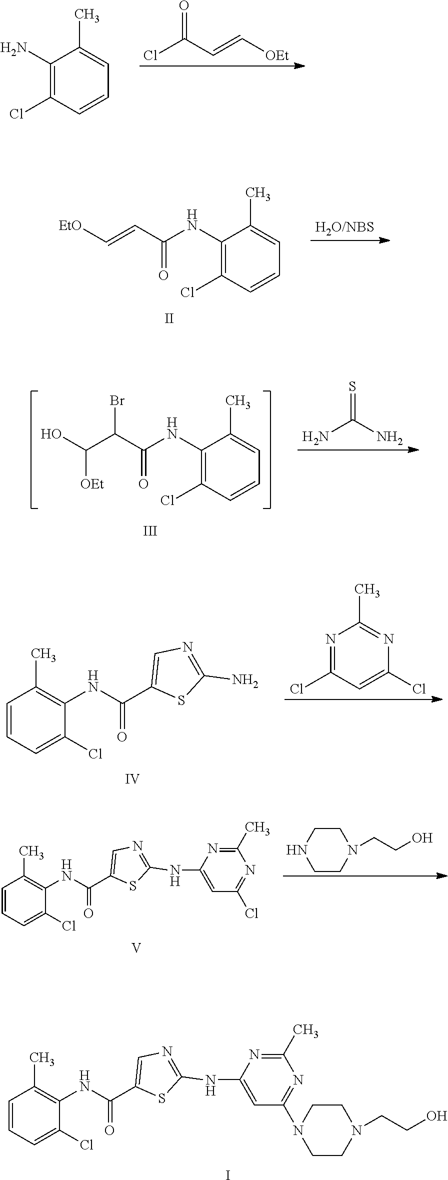

### Step 1: Preparation of (E)-N-(2-chloro-6-methylphenyl)-3-ethoxyacrylamide (Compound II)

2-chloro-6-methylaniline (34.8 g, 246 mmol) was dissolved in CH₂Cl₂ (300 mL), stirred at room temperature for dissolution, and then added with pyridine (18.6 g, 235 mmol). The resulting reaction liquid was cooled to 0° C. in an ice-water bath, and slowly added dropwise with (E)-3-ethoxyacryloyl chloride (39.6 g, 294 mmol), then the resulting reaction liquid was further stirred and reacted for 3 h in an ice-water bath. The solvent was removed by evaporation under reduced pressure, the residue was cooled to 0° C. in an ice-water bath, stirred and added with distilled water (180 mL) and stirred for an additional 30 min and filtered, the resulting filter cake was washed with distilled water (180 mL×2) and dried to give 55.8 g of (E)-N-(2-chloro-6-methylphenyl)-3-ethoxyacrylamide (II) as a white solid in a yield of 94.6%.

Melting point: 170 to 172° C.

Nuclear magnetic resonance detection:

¹H NMR (400 MHz, DMSO-d₆): 1.27 (t, 3H), 2.16 (s, 3H), 3.94 (q, 2H), 5.58 (d, 1H), 7.18-7.23 (m, 2H), 7.32 (d, 1H), 7.46 (d, 1H), 9.29 (s, 1H).

### Step 2: Preparation of 2-amino-N-(2-chloro-6-methylphenyl)-5-thiazolecarboxamide (Compound IV)

(E)-N-(2-chloro-6-methylphenyl)-3-ethoxyacrylamide (II) (51.6 g, 215 mmol) was dissolved in a mixed solution of distilled water (120 mL) and 1,4-dioxane (120 mL), placed in a low-temperature bath and cooled to −10° C., added with N-bromosuccinimide (42.2 g, 237 mmol), warmed to room temperature and stirred continuously for 3 h. Thiourea (16.5 g, 217 mmol) was added and the resulting reaction liquid was heated to 80° C. and then incubated with stirring for 2 h. The resulting reaction liquid was cooled to room temperature, slowly added dropwise with ammonia water to adjust the pH value of the solution to 8-9, concentrated under reduced pressure until a large amount of solid was precipitated. Distilled water (200 mL) was added to the resulting reaction liquid. The reaction liquid was cooled to 0° C. and filtered with suction. The resulting filter cake was washed with distilled water (200 mL×x2) and dried to give 55.1 g of 2-amino-N-(2-chloro-6-methylphenyl)-5-thiazolecarboxamide (IV) as a yellowish-brown solid in a yield of 95.7%.

Melting point: 207 to 209° C.

Nuclear magnetic resonance detection:

¹H NMR (400 MHz, DMSO-d₆): 2.21 (s, 3H), 7.24 (m, 2H), 7.36 (d, 1H), 7.69 (s, 2H), 7.87 (s, 1H), 9.66 (s, 1H).

### Step 3: Preparation of N-(2-chloro-6-methylphenyl)-2-[(6-chloro-2-methyl-4-pyrimidinyl)amino]-5 -thiazole carboxamide (Compound V)

2-amino-N-(2-chloro-6-methylphenyl)-5-thiazolecarboxamide (IV) (45.0 g, 168 mmol) was dissolved in anhydrous tetrahydrofuran (450 mL), added with 4,6-dichloro-2-methylpyrimidine (32.9 g, 202 mmol), and cooled below 10° C. with an ice-water bath. The resulting reaction liquid was slowly added with sodium tert-butoxide (56.3 g, 586 mmol), continued to be stirred and reacted for 3 h. The pH value of the solution was adjusted to 6 with hydrochloric acid, crystal was precipitated. The reaction mixture was continued to be stirred for 1.5 h, and filtered. The resulting filter cake was washed with distilled water (100 mL) and dried to give 59.3 g of N-(2-chloro-6-methylphenyl)-2-[(6-chloro-2-methyl-4-pyrimidinyl)amino]-5 -thiazole carboxamide (V) as a faint yellow solid in a yield of 89.5%.

Melting point: >300° C.

Nuclear magnetic resonance detection:

¹H NMR (400 MHz, DMSO-d₆): 2.23 (s, 3H), 2.57 (s, 3H), 6.93 (s, 1H), 7.27 (m, 2H), 7.39 (d, 1H), 8.28 (s, 1H), 10.00 (s, 1H), 12.20 (s, 1H).

### Step 4: Preparation of N-(2-chloro-6-methylphenyl)-2-{6-[4-(3-hydroxyethyl)-1-piperazinyl]-2-methyl-4-pyrimidinylamino}-5-thiazolecarboxamide (Compound I)

N-(2-chloro-6-methylphenyl)-2-[(6-chloro-2-methyl-4-pyrimidinyl)amino]-5 -thiazolecarboxamide (V) (52.5 g, 133 mmol) was dissolved in dimethyl sulfoxide (500 mL), and added with 1-(2-hydroxyethyl)piperazine (60.9 g, 265 mmol) and N, N-diisopropylethylamine (34.7 g, 267 mmol). The resulting reaction liquid was heated to 80° C. and then reacted under incubation with stirring for 1 h. The resulting reaction liquid was added with distilled water (1000 mL), cooled to 15° C. in an ice-water bath, and slowly stirred for 1 h for crystallization, filtered and dried to give 61.2 g of faint yellow solid. The solid was added to 80% aqueous ethanol solution (650 mL), heated to 80° C., incubated with stirring until the solid was completely dissolved, naturally cooled to 15° C., and incubated with stirring for 1 h, filtered and dried to give 50.6 g of N-(2-chloro-6-methylphenyl)-2-{6-[4-(3-hydroxyethyl)-1-piperazinyl]-2-methyl-4-pyrimidinylamino}-5-thiazolecarboxamide (I) as a white solid in a yield of 75.2%.

HPLC Purity: 99.4%

Melting point: 282 to 283° C.

Nuclear magnetic resonance detection:

¹H NMR (400 MHz, DMSO-d₆): 2.24 (s, 3H), 2.42 (s, 3H), 2.46 (t, 2H), 2.50 (t, 4H), 3.50 (m, 4H), 3.54 (q, 2H), 4.41 (t, 1H), 6.05 (s, 1H), 7.23 (t, 1H), 7.27 (d, 1H), 7.39 (d, 1H), 8.21 (s, 1H), 9.85 (s, 1H), 11.43 (s, 1H).

### EXAMPLE 2

### Pharmacodynamic Test of Compound I Against Amyloid-β Protein Mouse Cerebral Cortical Neuron Toxicity

The compound JKF-027 used in the test of the present invention was prepared according to the method reported in the literature: WO2012/103282 (Publication Date: Aug. 2, 2012).

1) Test purpose

Amyloid-β 42 oligomer was used to induce fetal mouse primary isolated cerebral cortical neuron injury to test the effect of Compound I against Aβ42 protein toxicity.

2) Test Material

(1) Basic information of sample for test

| Name or | Sample for test | | Model drug |
|---|---|---|---|
| code name: | JKF-027 | Compound I | Aβ42 |
| Storage | −20° C. keep | −20° C. keep | −20° C. keep |
| conditions: | in dark | in dark | in dark |
| | place | place | place |
| Preparation | Diluted by NM | Diluted by NM | PBS + |
| method: | medium | medium | 0.5%DMSO |
| Mark after | The NM medium | The NM medium | The PBS + 0.5% |
| preparation: | dilution dose | dilution | DMSO dose |
| | group is | dose group | group is |
| | labeled with | is labeled | labeled with |
| | a white | with a | a white |
| | label and marked | yellow label and | label and marked |
| | with test | marked with test | with test number, |
| | number, test | number, test | test substance |
| | substance | substance | name and |
| | name and | name and | concentration, |
| | concentration, | concentration, | preparation |
| | preparation | preparation | volume, |
| | volume, | volume, | storage |
| | storage | storage | conditions, |
| | conditions, | conditions, | validity period, |
| | validity period, | validity period, | person in |
| | person in | person in | charge and |
| | charge and | charge and | preparation |
| | preparation | preparation | date, etc. |
| | date, etc. | date, etc. | |
| Temporary | 4° C. | 4° C. | 4° C. |
| storage | | | |
| conditions after | | | |
| preparation: | | | |
| Validity period | 24 h | 24 h | 24 h |
| after preparation: | | | |

(2) Preparation of Sample for Test

Preparation method: Compound I and JKF-027 were diluted to 7 concentrations.

Temporary storage conditions and Validity period after preparation: the solution was used right after it was ready.

3) Test Method

(1) Primary Isolation of Fetal Mouse Neurons

Test preparation: sterilized surgical instrument, pipette, ice pack, culture dish, 70% alcohol and cotton ball, centrifuge tube, 12 well plate (coated), pancreatic enzyme, HBSS solution, DNase, growth medium, and maintenance medium.

Test operation: The uterus of the pregnant mouse with pregnancy of 17 days was taken out, and the embryos were separated one by one. The cortex of the fetal mouse was taken out, placed in HBSS and washed several times. The enzyme was added for digestion treatment and then the supernatant was discarded. The growth medium was added. The culture dish was tilted, slowly blown with a 1 mL pipette, and after resting, the upper single suspended cells were transferred to a centrifuge tube, and the above operation was repeated three times.

(2) Pre-Test: Toxicity Concentration Test for Aβ42-Induced Neuron Injury

On the 6th day of neuron culture, cells were divided into 6 groups with Aβ42 concentrations of 0, 10, 20, 40, 80 and 160 μmol/L, respectively, and mixture of Aβ42 monomer and oligomer (H4) was added to each group for a duration of 24 hours with one replicate well. Twelve cell culture wells were used in one test, and the test was repeated one time. The test grouping is shown in Table 1.

TABLE 1
Modeling concentration grouping table (μmol/L)

| | Concentration | | | | | | | | | | | |
|---|---|---|---|---|---|---|---|---|---|---|---|---|
| Substance | 0 | | 10 | | 20 | | 40 | | 80 | | 160 | |
| Mixture of Aβ42 | H4 | complex | H4 | complex | H4 | complex | H4 | complex | H4 | complex | H4 | complex |
| monomer and | | | | | | | | | | | | |
| oligomer | | | | | | | | | | | | |

According to the results of the test, WST-8 was used to measure cell viability, and Aβ42 concentration with a neuron viability of 30% to 60% was screened as formal molding concentration to determine neuroprotective activity of JKF-027.

(3) Neuroprotective Activity Assay of Compound I

Sample adding: It could be seen from the cell viability that when the concentration was higher than 80 μmol/L, the concentration was so high that the molding drug aggregated with each other to form a colloidal substance, which influenced the interaction between the proteins, resulting in a decrease in polymerization efficiency of oligomers and a decrease in cytotoxic effect of Aβ42 at a high concentration. However, when the concentration was lower than 10 μmol/L, the concentration of the molding drug was low, and the oligomerization efficiency was low in the same polymerization time, resulting in a decrease in cytotoxicity. The cell viability in the concentration range of 10 μmol/L to 80 μmol/L could meet the pre-set requirements. Since the content of Aβ42 in human is very low, the inventors chose the concentration of the molding drug of 10 μmol/L to establish a cell injury model. On the 6th day of neuron culture, the same concentration of Aβ42 and different concentrations of different tested drugs were added and cultured for 24 h, and the cell activity was detected with WST-8. And a complex hole and two negative control holes and two normal control holes were set. The test was repeated 2 times. The test grouping is shown in Table 2.

TABLE 2
Drug concentration grouping table (μmol/L)

| | Concen- | | Concen- | | Concen- | | Concen- | | Concen- | | Concen- | | Concen- | |
|---|---|---|---|---|---|---|---|---|---|---|---|---|---|---|
| | tration 1 | | tration 2 | | tration 3 | | tration 4 | | tration 5 | | tration 6 | | tration 7 | |
| JFK-027 | 1000 | complex | 100 | complex | 10 | complex | 1.0 | complex | 0.1 | complex | 0.01 | complex | — | — |
| Compound I | 1000 | complex | 100 | complex | 10 | complex | 1.0 | complex | 0.1 | complex | 0.01 | complex | 0.001 | complex |

(4) Detection of Cell Viability with WST-8

A corresponding amount of WST-8 solution was added to each well.

The culture plate was incubated in an incubator for 2-4 hours.

The absorbance at 450 nm was measured with a microplate reader. 4) Cell Activity Detection

Cell activity *(%)=[A (dosing)−A (blank)]/[A (without dosing)−A (blank)]×100%

A (dosing): the absorbance of well having cells, WST solution and drug solution

A (blank): the absorbance of well having medium and WST solution and no cells

A (without dosing): the absorbance of well having cells and WST solution and no drug solution

*Cell activity: cell proliferation activity or cytotoxic activity

5) Test Results

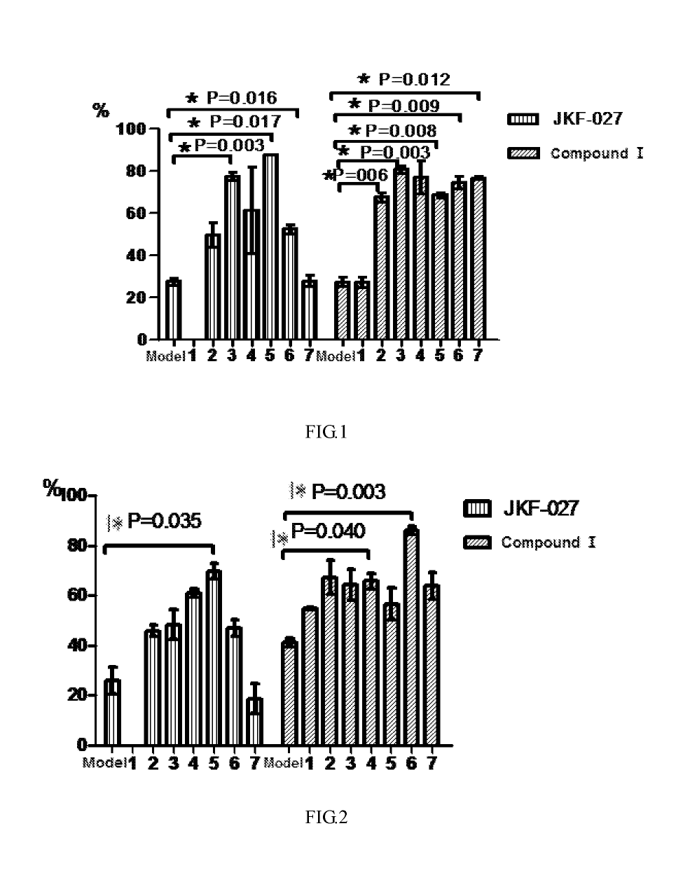

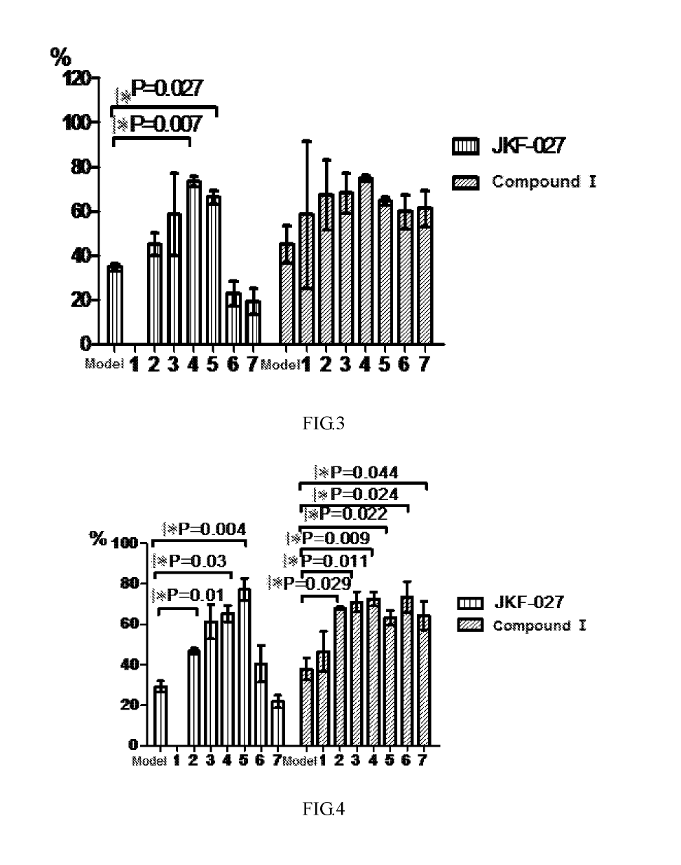

The results of the first independent test are shown in Table 3 and FIG. 1. The results of the second independent test are shown in Table 4 and FIG. 2. The results of the third independent test are shown in Table 5 and FIG. 3. The mean values of the three independent tests are shown in Table 6 and FIG. 4. From the test results, the three tests have a very good repeatability, and the result is more convincing. The abscissa represents different administration concentrations, 1 represents 0.001 μM; 2 represents 0.01 μmol/L; 3 represents 0.1 μmol/L; 4 represents 1.0 μmol/L; 5 represents 10 μmol/L; 6 represents 100 μmol/L; and 7 represents 1000 μmol/L. The anti-Aβ42 protein toxicity curves of different tested drugs with different concentrations show a trend of first rising and then declining, and all have an effective dose interval. Compared with the model group, JKF-027 has an optimum anti-toxic concentration of 0.1-10 μmol/L. Compared with JKF-027, Compound I has a wide range of anti-toxic effective concentration, anti-toxic effect of multiple effective concentrations with significant difference compared with that of model group, no significant difference between effective concentrations, and has obvious anti-toxic effect in the concentration of 0.01-1000 μmol/L.

It can be seen from the results of three replicate tests, the values of the three tests are basically the same, and the trends of cell tests of drugs show a normal peak curve, and both tested drugs are effective. JKF-027 has an optimum anti-toxic concentration of 0.1-10 μmol/L. Compared with JKF-027, Compound I has a wide range of anti-toxic effective concentration, anti-toxic effect of multiple effective concentrations with no significant difference, and anti-toxic effect in the concentration of 0.01-1000 μmol/L with significant difference compared with that of model group, therefore has a much better anti-Alzheimer's effect than JKF-027.

TABLE 3
Cell viability data in the first independent test

| | Model | 0.001 μM/L | 0.01 μM/L | 0.1 μM/L | 1 μM/L |
|---|---|---|---|---|---|
| JKF-027 | 27.44 | | 49.59 | 77.44 | 61.59 |
| Compound I | 27.44 | 26.98 | 69.31 | 80.78 | 76.90 |

| | 10 μM/L | 100 μM/L | 1000 μM/L |
|---|---|---|---|
| JKF-027 | 87.60 | 51.83 | 27.85 |
| Compound I | 68.61 | 74.43 | 76.72 |

TABLE 4
Cell viability data in the second independent test

| | Model | 0.001 μM/L | 0.01 μM/L | 0.1 μM/L | 1 μM/L |
|---|---|---|---|---|---|
| JKF-027 | 25.88 | | 45.88 | 48.40 | 61.01 |
| Compound I | 41.35 | 54.70 | 67.22 | 64.25 | 65.73 |

| | 10 μM/L | 100 μM/L | 1000 μM/L |
|---|---|---|---|
| JKF-027 | 69.75 | 46.89 | 18.66 |
| Compound I | 56.51 | 86.16 | 63.92 |

TABLE 5
Cell viability data in the third independent test

| | Model | 0.001 μM/L | 0.01 μM/L | 0.1 μM/L | 1 μM/L |
|---|---|---|---|---|---|
| JKF-027 | 34.67 | | 45.00 | 58.33 | 73.33 |
| Compound I | 44.97 | 58.28 | 67.46 | 68.05 | 74.70 |

| | 10 μM/L | 100 μM/L | 1000 μM/L |
|---|---|---|---|
| JKF-027 | 74.50 | 22.83 | 19.17 |
| Compound I | 64.50 | 59.62 | 52.37 |

TABLE 6
Mean value of cell viability based on three independent replicates

| Number of | Normal | Modeling | | | | |
|---|---|---|---|---|---|---|
| sample for test | group | group | 0.001 μM | 0.01 μM | 0.1 μM | 1.0 μM |
| JKF-027 | 100% | 29.33% | — | 46.83% | 61.39% | 65.31% |
| Compound I | 100% | 37.88% | 46.65% | 67.99% | 71.02% | 72.44% |
| Number of | | | 10 μM | 100 μM | 1000 μM | |
| sample for test | | | | | | |
| JKF-027 | | | 77.28% | 40.52% | 21.89% | |
| Compound I | | | 63.20% | 73.40% | 64.34% | |

1. Use of a Src protein inhibitor in the manufacture of a medicament for:
(1) treatment and/or prophylaxis of Alzheimer's disease or senile dementia;
(2) treatment and/or prophylaxis of nervous system degenerative diseases;
(3) improvement of cognitive disorder or learning and memory disorder;
(4) inhibition of Aβ42 cytotoxicity;
(5) treatment and/or prophylaxis of vascular dementia or vascular cognitive impairment;
(6) treatment and/or prophylaxis of cholinergic neuron degenerative diseases.

2. The use of claim 1, wherein the Src protein inhibitor is a Src/Abl dual-acting inhibitor.

3. The use of claim 1, wherein the Src protein inhibitor is selected from dasatinib or a pharmaceutically acceptable salt thereof, bosutinib or a pharmaceutically acceptable salt thereof.

4. Use of a compound of formula I or a pharmaceutically acceptable salt thereof, a solvate thereof or a solvate of the salt, an ester or ether thereof, a precursor or metabolite thereof in the manufacture of a medicament for: 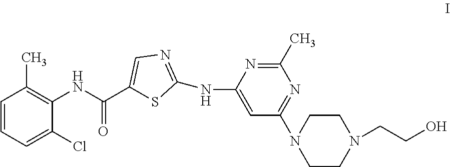 [SMILES: Cc1nc(Nc2ncc(C(=O)Nc3c(C)cccc3Cl)s2)cc(N2CCN(CCO)CC2)n1]
(1) treatment and/or prophylaxis of Alzheimer's disease or senile dementia;
(2) treatment and/or prophylaxis of nervous system degenerative diseases;
(3) improvement of cognitive disorder or learning and memory disorder;
(4) inhibition of Aβ42 cytotoxicity;
(5) treatment and/or prophylaxis of vascular dementia or vascular cognitive impairment;
(6) treatment and/or prophylaxis of cholinergic neuron degenerative diseases.

5. The use of claim 4, wherein the compound of formula I is a compound of formula I in the form of free base, or a pharmaceutically acceptable salt thereof.

6. The use of claim 4, wherein the pharmaceutically acceptable salt is hydrochloride, hydrobromide, sulfate, phosphate, nitrate, acetate, propionate, succinate, glycollate, stearate, lactate, malate, tartrate, citrate, ascorbate, pamoate, maleate, hydroxymaleate, phenylmaleate, glutamate, benzoate, salicylate, fumarate, tosylate, mesylate, oxalate and isethionate.

7. The use of claim 4, wherein the solvate or the solvate of the salt refers to a hydrate of the compound of the formula I or a hydrate of the salt of the compound of formula I.

8. The use of claim 4, wherein the medicament comprises the compound of formula I or pharmaceutically acceptable salt thereof, solvate thereof or solvate of the salt, ester or ether thereof, precursor or metabolite thereof, and one or more pharmaceutically acceptable additives.

9. The use of claim 4, wherein the medicament is an oral preparation, intramuscular, intraperitoneal, intravenous, ICV, intracisternal injection or infusion preparations, subcutaneous injection or implantation preparations, inhalation spray, nasal, vaginal, rectal, sublingual or topical preparations.

10. The use of claim 4, wherein in the medicament, the compound of formula I or pharmaceutically acceptable salt thereof, solvate thereof or solvate of the salt, ester or ether thereof, precursor or metabolite thereof is contained in an amount of 0.1-99.9%, preferably 1-90%, more preferably 5-80%, and most preferably 10-70% by mass percent.
